(19) United States
(12) Patent Application Publication
(10) Pub. No.: US 2025/0345090 A1
(43) Pub. Date: Nov. 13, 2025

(54) MEDICAL DEVICE FOR GUIDING A NEEDLE TO A PUNCTURE POSITION

(71) Applicant: Xabier ERRAZKIN TELLETXEA, Astigarraga (ES)

(72) Inventor: Xabier ERRAZKIN TELLETXEA, Astigarraga (ES)

(21) Appl. No.: 19/276,838

(22) Filed: Jul. 22, 2025

Related U.S. Application Data
(63) Continuation of application No. PCT/ES2023/070037, filed on Jan. 24, 2023.

(51) Int. Cl. A61B 17/34; A61B 90/00

(52) U.S. Cl. CPC A61B 17/3403; A61B 90/06; A61B 2017/3407

(57) ABSTRACT

A medical device for guiding a tip of a puncture device to a puncture position. The medical device includes a guiding platform that facilitates longitudinal movement of the tip to a puncture position. The guiding platform is coupled to a base body that is configured to be supported on a puncture area. The guiding platform is pivotably attached to the base body such that the guiding platform can be fixed at different angles of inclination with respect to the base body.

### CROSS-REFERENCE TO RELATED APPLICATIONS

This application relates to and claims the benefit and priority to International Application No. PCT/ES2023/070037, filed Jan. 23, 2023, which is incorporated herein by reference in its entirety.

### FIELD

The present invention relates to puncture needle guiding devices for human or animal use.

### BACKGROUND

The use of sharp-pointed medical devices, such as puncture needles, scalpels, or a trocar, in humans or animals requires the practitioner to have the skill and dexterity to perform puncture manually. Depending on the type of needle, the angle of puncture is a determining factor in reaching the internal area of interest, avoiding possible undesired tears and injuries to the patient. Sometimes, guiding by means of visualizing the veins or areas where puncture is to be performed through ultrasound devices is also required, which further complicates the puncturing process.

U.S. Pat. No. 4,403,987A describes a device for supporting a hypodermic syringe and for facilitating injection of the needle of the hypodermic syringe into an arm or leg. The device includes a syringe supporting element and guiding means for supporting the movement of said element between a first position in which a needle supported on the syringe supporting member is retracted and a second injecting position. The guiding means include a pair of guide rods for supporting the syringe supporting element for slidable movement between the first position and the injecting position, and means for supporting the guiding means and for pinching flesh adjacent the syringe needle.

### SUMMARY

Disclosed is a medical device for guiding a needle to a puncture position.

The medical device of the invention comprises a guiding platform to allow a longitudinal movement of the needle on the guiding platform to a puncture position, the guiding platform being coupled to a base body which is supported on a puncture area, wherein the guiding platform is pivotably attached to the base body, such that said guiding platform can be fixed at different angles of inclination with respect to the base body

The device of the invention is configured to be used manually by the practitioner, allowing the practitioner to guide the needle over the puncture area of the body in a controlled manner, allowing the pointed device to be guided at a specific angle to avoid collateral damage due to undesired tearing of the vein.

In addition to being structurally simple and easy to manufacture, the device can be used together with visualization devices, such as an ultrasound device, which is extremely useful in ultrasound-directed vascular punctures.

These and other advantages and features of the invention will become apparent in view of figures and detailed description of the invention.

### BRIEF DESCRIPTION OF THE DRAWINGS

FIG. 1 shows a perspective view an embodiment of a device for guiding a needle to a puncture position.

FIGS. 2 and 3 show a perspective view of the device of FIG. 1 attached to an ultrasound device in two different usage positions.

FIG. 4 shows a partial perspective view of an embodiment of a guiding platform.

FIG. 5 shows a section view of a guiding platform, a support plate slidable on the guiding platform and a part of a needle assembly positioned on the support according to an embodiment.

FIG. 6 shows a section view of a guiding platform, a support plate slidable on the guiding platform and a part of a needle assembly positioned on the support according to another embodiment.

FIGS. 7A-C shows a schematic depiction of different angular positions of the guiding platform with respect to a puncture area with the base body in a first position.

FIGS. 8A-C shows a schematic depiction of different angular positions of the guiding platform with respect to a puncture area with the base body in a second position.

FIG. 9 shows a perspective view of a guiding platform according to one embodiment.

### DETAILED DESCRIPTION

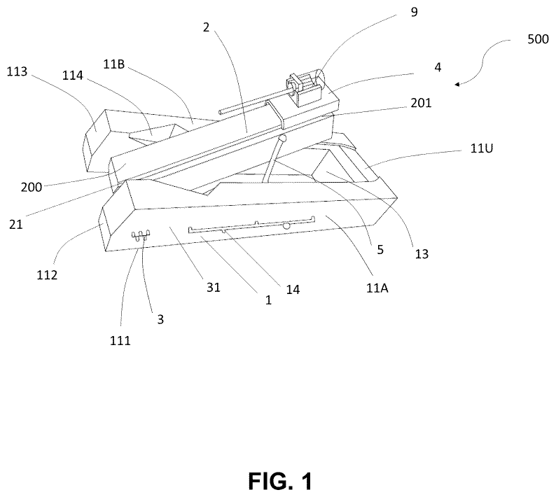

As shown in FIG. 1, the device 500 for guiding a needle to a puncture position comprises a guiding platform 2 which allows a longitudinal movement of the needle on the guiding platform 2 from a retracted position 201 to a puncture position 200, the guiding platform 2 being coupled to a base body 1 configured for being supported on a puncture area, with the guiding platform 2 being pivotably attached to the base body 1, such that said guiding platform 2 can pivot and be fixed at different angles of inclination with respect to the base body 1. The user will be able to tilt or pivot the guiding platform at a specific angle with respect to the puncture area, depending on the angle of puncture required.

In the context of the invention, puncture area refers to the area of the animal or human body close to the area where the puncture should be made.

In the context of the invention, angle of puncture refers to the angle the pointed or puncture device, for example, the needle, must adopt depending on the puncture to be performed, i.e., intramuscular, subcutaneous, endovenous, or intradermal.

In the context of the invention, puncture position is understood as when the puncture device, more specifically the pointed element of the puncture device, for example, a needle tip, is in a contact position with the area where the puncture should be made.

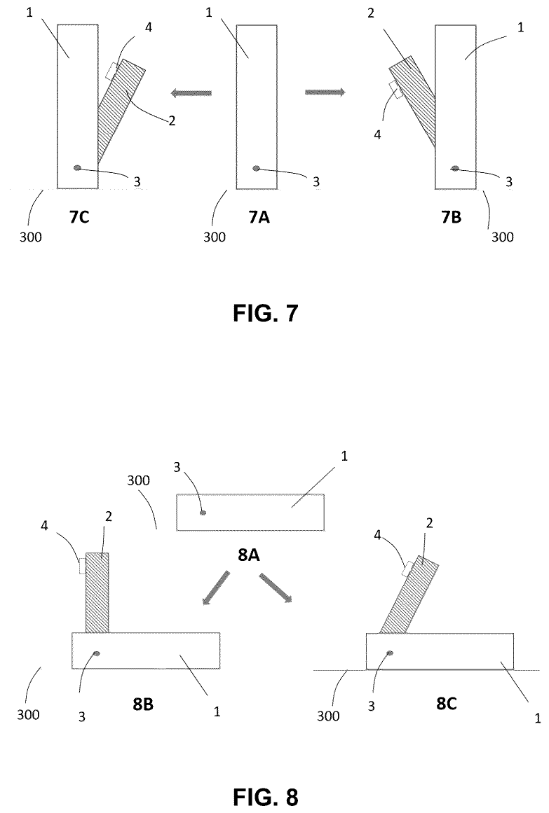

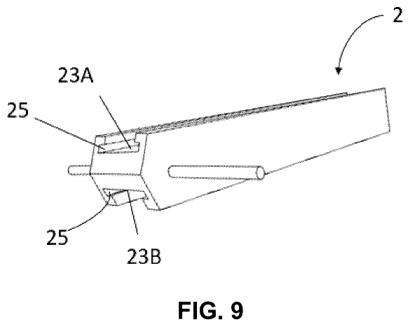

In one embodiment, the base body 1 can be arranged on the puncture area in a first position in which the guiding platform 2 is arranged parallel to the puncture area when it is arranged with an inclination of 0°, i.e., the base body 1 can be arranged on the puncture area in a first position in which the guiding platform 2 is arranged parallel to the puncture area 300 in the case where the guiding platform is arranged aligned with the base body 1, as shown in FIG. 8A. In another embodiment, the base body is arranged on the puncture area in a second position in which the guiding platform 2 is arranged perpendicular to the puncture area when said guiding platform is arranged with an inclination of 0°, i.e., the guiding platform 2 is arranged perpendicular to the puncture area in the case where said guiding platform is arranged aligned with the base body 1, as shown in FIG. 7 (see position 7A). In a preferred embodiment, the device can be arranged in a first position or a second position, according to practitioner's needs.

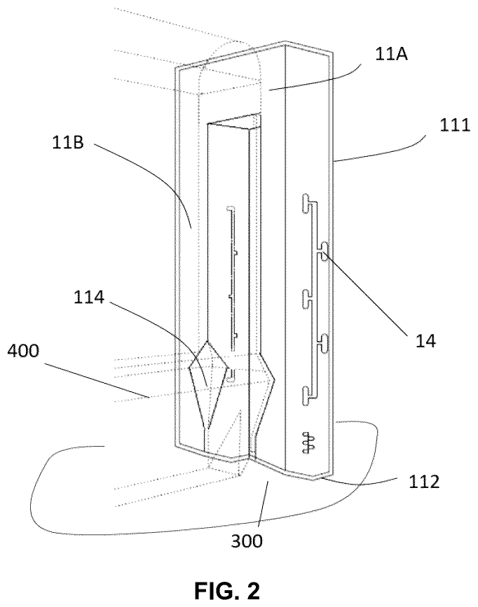

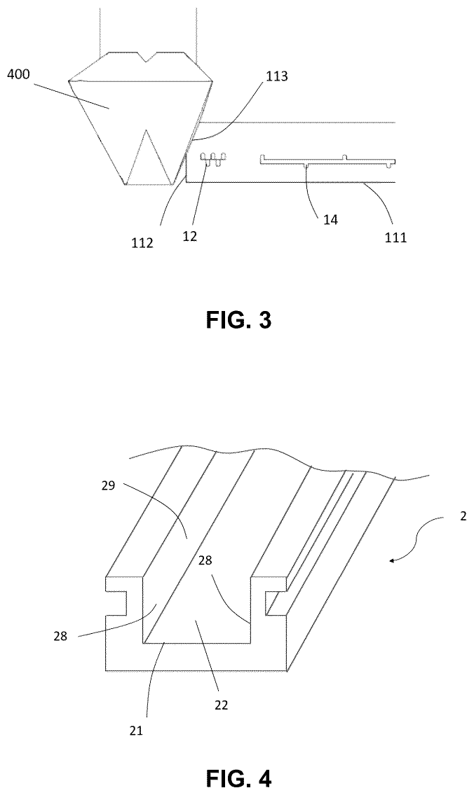

To better support the base body 1 on the puncture area 300, as shown in FIGS. 1 to 3, the base body 1 comprises in this embodiment at least one contact surface 111, 112 for being supported on a support surface of the puncture area 300. For example, contact surface 111 is used in the embodiment shown in FIG. 3, whereas surface 112 is used in the embodiment shown in FIG. 2. In another embodiment, the base body 1 comprises a second contact surface 113, 114 for being supported on a surface of an ultrasound imaging device, such as a probe of an ultrasound device 400 in FIG. 2 (drawn with a dotted line) and FIG. 3. This contact surface 113, 114 is useful when imaging by means of an ultrasound device is required to guide the puncture within the human body. This feature is useful in both the first position and the second position of the base body 1 described above, as shown in FIGS. 2 and 3. The guiding platform has been omitted in FIG. 2 as it seeks to illustrate mainly the position of attachment with the ultrasound device.

In terms of the angles at which the guiding platform 2 pivots and is fixed, in one embodiment, the guiding platform 2 pivots and is fixed at an angle of at least between 0° and 180°, preferably between 0° and 90°, more preferably between 5° and 90°, with respect to the base body 1, when the base body 1 is arranged on the puncture area in the first position 8A. In another embodiment, the platform pivots and is fixed at an angle of at least between 0° and 90° or between 0° and-90°, more preferably at least between 5° and 90° or between −5° and −90°, with respect to the base body 1, when the base body 1 is arranged on the puncture area in the second position. In another embodiment, when it is not in any position, the platform pivots at an angle between 0° and 360°.

In one embodiment, the base body 1 comprises a rotating shaft 3, with one end 31 of the guiding platform 2 being coupled to said rotating shaft 3, the rotating shaft being configured to pivot the guiding platform 2 at a specific angle.

In one embodiment, the base body 1 comprises securing areas 12 for the rotating shaft 3 configured so that the guiding platform 2, in a specific angle of inclination, guides the needle to a specific area of the puncture area, this specific area being the area where the pointed element will perform puncture. FIG. 3 illustrates an embodiment in which the base body 1 comprises notches 12 which secure the rotating shaft 3. Depending on the specific area to be punctured, the user will be able to fix the shaft in one notch or another.

In a preferred embodiment, as illustrated in FIGS. 1, 7, and 8, the guiding platform 2 pivots between a first position in which the guiding platform 2 is arranged in a housing 13 of the base body 1, thus being aligned with the base body 1, and a second position in which the guiding platform 2 is pivoted partially out of the base body 1. In the case of FIG. 7, the base body 1 is supported on a contact surface 112, such that the housing 13 is perpendicular to the puncture area. In this embodiment, the guiding platform can adopt an angle of between 0 and 90° and between 0 and −90°, the last being useful for use thereof attached to probes of ultrasound devices. In the case of FIG. 8, the base body 1 is supported on a contact surface 111, such that the housing 13 is horizontal to the puncture area. In this embodiment, the guiding platform can adopt an angle of between 0 and 180°. FIG. 8 shows the transition to 90° with respect to the contact area.

In an embodiment of the invention as shown in FIGS. 1 and 2, the base body 1 comprises two parallel supports 11A, 11B comprising contact surfaces, with the rotating shaft 3 being arranged between both side supports 11A, 11B, preferably, between the ends of both side supports 11A, 11B. The housing 13 for the guiding platform 2 is arranged between both side supports. Additionally, the base body may have a third support 11U perpendicular to the side supports, the base body 1 adopting a U shape.

In another embodiment not shown in the figures, the base body 1 has two supports, said supports being attached in an L shape, with the rotating shaft 3 being arranged at a free end of one of the supports. The housing 13 is arranged between both supports.

In one embodiment, the guiding platform 2 is fixed by fixing means 5 which fix the guiding platform 2 with the base body 1 at a desired angle of inclination. Preferably, these fixing means are at least one rod which attaches the guiding platform 2 with the base body 1. The rod can be attached at different points 14 of the base body 1 such that the guiding platform 2 can be fixed in different angular positions. FIG. 3 shows several anchoring notches or points 14 arranged in the base body 1, with the notches identifying the envisaged degree of inclination, and allowing the anchoring of one end of the rod to the notch, depending on the required angular position of the guiding platform 2, being preferably at 10°, 15°, 25°, 45°, and 90° with respect to the puncture area.

In another embodiment not shown in the figures, the fixing means consist of a gearwheel shaft, where this shaft may coincide with the rotating shaft 3.

With respect to the guiding platform 2, in a preferred embodiment, the platform comprises guiding means 29 and a surface 22 for sliding between a retracted position 201 and a puncture position 200 of the puncture needle. In one embodiment shown in FIG. 4, the guiding platform 2 comprises a rectangular prism comprising at least one longitudinal channel 21 with a base 22 and walls 28, with the base 22 acting as a sliding surface and the walls 28 as guiding means 29.

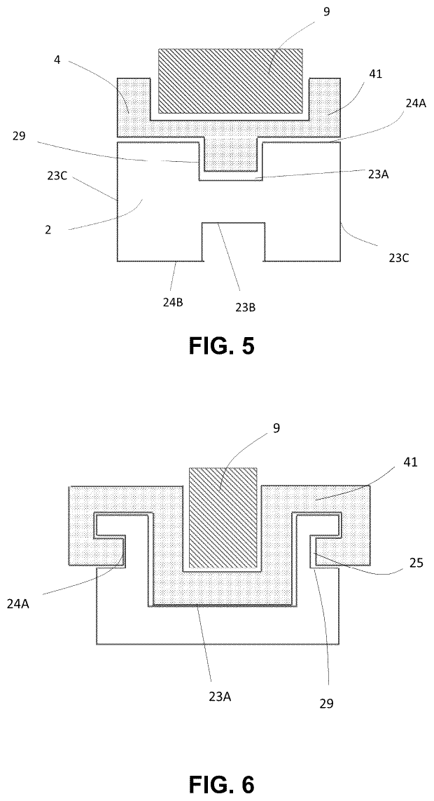

In another embodiment illustrated in FIG. 5, the guiding platform 2 comprises a support 4, preferably a support plate 41, configured to retain at least one part 9 of the needle, for example, a rear chamber of a needle, the support 4 being arranged on or engaged in the guiding means 29, said guiding means 29 allowing the sliding of the support 4 in the longitudinal direction on said guiding means 29 between a retracted position 201 and a puncture position 200. In a preferred embodiment, the guiding means 29 comprise a longitudinal surface 24A, 24B and at least one longitudinal groove or recess 23A, 23B which is coupled to at least one portion of the support 4, slidingly retaining the support 4 between a retracted position 201 and a puncture position 200 of the support 4. FIGS. 5 and 6 show an example of two embodiments in which the guiding platform 2 comprises a rectangular prism comprising at least one longitudinal recess 23A on face 24A of the prism. Faces 23C adjacent to face 24A may comprise a longitudinal groove or recess 25, as shown in FIG. 6. In another embodiment, the guiding means 29 comprise two longitudinal opposite surfaces 24A, 24B, with their corresponding recesses 23A, 23B, 25 or grooves. The user will therefore be able to choose to place the support 41 on face 24A or on face 24B, depending on whether they choose a positive angle (between 0° and 90°) or a negative angle (between 0° and −90°).

Preferably, the guiding platform 2, in the different embodiments thereof, allows movement of the needle from a retracted position 201 to a puncture position 200 and vice versa, being extremely useful in guiding catheter-type needles since, in these cases, once the catheter has been introduced with the needle, the needle must be removed carefully, avoiding the tearing of vein or tissue.

To better control the sliding of the needle or the support comprising the needle on the guiding platform 2, the guiding platform 2 may comprise a surface which generates a resistance to the free sliding of the needle or the support on the guiding platform, so it is necessary to exert a thrust force with the hand or finger on the needle. By way of example, this resistance can be achieved by means of a coarse surface or adapting the clearance of the recesses or grooves described above.

In one embodiment, the guiding platform 2 comprises an element for measuring the distance traveled by the needle during puncture. This feature, for example, is useful in ultrasound-directed punctures in which needle angulation and advancement control within the animal or human body is critical. The ultrasound device allows identifying the target structure to be punctured, as well as the depth of the target structure. The device of the invention allows fixing the angulation and knowing the distance traveled by the needle within the human body. In a preferred embodiment, the element for measuring is a meter. By way of example, the guiding means 29 and/or the surface 22 of the guiding platform may comprise distance marks such as the distance marks of a ruler. These marks allow the user to know the distance traveled by the puncture needle.

Although the device has been described for a needle, it can also work with any other sharp-pointed instrument, such as a scalpel or a trocar.

Embodiments are disclosed in the clauses that follow.
Clause 1. A medical device for guiding a needle to a puncture position, comprising a guiding platform (2) to allow a longitudinal movement of the needle on the guiding platform (2) to a puncture position (200), the guiding platform (2) being coupled to a base body (1) which is supported on a puncture area (300), wherein the guiding platform (2) is pivotably attached to the base body (1), such that said guiding platform (2) can be fixed at different angles of inclination with respect to the base body (1), the base body (1) can be arranged on the puncture area in a first position in which the guiding platform (2) is arranged parallel to the puncture area when it is arranged with an inclination of 0°, and in a second position in which the guiding platform (2) is arranged perpendicular to the puncture area when said guiding platform is arranged with an inclination of 0°, wherein the guiding platform (2) pivots and is fixed at an angle of between 0° and 180° with respect to the base body (1) when the base body (1) is arranged on the puncture area in the first position, and wherein the guiding platform (2) pivots and is fixed at an angle of between 0° and 90° or 0° and −90° with respect to the base body (1) when the base body (1) is arranged on the puncture area in the second position.
Clause 2. The device according to clause 1, wherein the base body (1) comprises at least one contact surface (111, 112) for being supported on a support surface of the puncture area (300).
Clause 3. The device according to clause 1 or 2, wherein the base body (1) comprises a contact surface (113) for being supported on a surface of a probe of an ultrasound device.
Clause 4. The device according to any of the preceding clauses, wherein the base body (1) comprises a rotating shaft (3), with one end (21) of the guiding platform (2) being coupled to said rotating shaft (3), the rotating shaft being configured to pivot the guiding platform (2) at a specific angle with respect to the base body (1).
Clause 5. The device according to clause 4, wherein the base body (1) comprises securing areas (12) for the rotating shaft (3) configured so that the guiding platform (2), at a specific angle of inclination, guides the needle to a specific area of the puncture area (300).
Clause 6. The device according to clause 4 or 5, wherein the base body (1) comprises two parallel supports (11A, 11B) comprising the contact surface, with the rotating shaft (3) being arranged between both side supports (11A, 11B).
Clause 7. The device according to clause 4 or 5, wherein the base body (1) has two supports comprising the contact surface, said supports being attached in an L shape, with the rotating shaft (3) being arranged on one of the supports.
Clause 8. The device according to any of the preceding clauses, wherein the guiding platform (2) pivots between a first position in which the guiding platform (2) is arranged in a housing (13) of the base body (1), and a second position in which the guiding platform (2) is pivoted partially out of the base body (1).
Clause 9. The device according to any of the preceding clauses, wherein the guiding platform (2) is fixed by fixing means (5), the fixing means (5) comprising at least one rod which attaches the guiding platform (2) with the base body (1) wherein the rod can be attached at different points (14) of the base body (1), such that the guiding platform (2) can be fixed in different angular positions.
Clause 10. The device according to any of the preceding clauses, wherein the guiding platform (2) comprises guiding means (29) and at least one surface (22) for sliding between a retracted position (201) and a puncture position (200) of the puncture needle.
Clause 11. The device according to any of the preceding clauses, wherein the guiding platform (2) comprises a rectangular prism comprising at least one longitudinal channel (21) with a base (22) and walls (28), with the base (22) acting as a sliding surface and the walls (28) as guiding means (29).
Clause 12. The device according to any of clauses 1 to 10, wherein the guiding platform (2) comprises a support (4), preferably a support plate (41), configured to retain at least one part of a needle (9), the support (4) being arranged on the guiding means (29), said guiding means (29) allowing the sliding of the support (4) in the longitudinal direction on said guiding means (29) between a retracted position (201) and a puncture position (200).
Clause 13. The device according to clause 12, wherein the guiding means (29) comprise a longitudinal surface (24A) and at least one longitudinal groove or recess (23A) which is coupled to at least one portion of the support (4), slidingly retaining the support (4), between a retracted position (201) and a puncture position (200) of the support plate (41).
Clause 14. The device according to clause 13, wherein the guiding means (29) comprise two longitudinal opposite surfaces (24A, 24B).
Clause 15. The device according to any of the preceding clauses, wherein the guiding platform (2) comprises an element for measuring the distance traveled by the needle during puncture, preferably a meter.

1. A medical device for guiding a pointed element of a puncture device to a puncture position, the medical device comprising:
a base body that includes a first contact surface facing a first direction and a second contact surface facing a second direction different than the first direction, each of the first and second contact surface being configured to rest on a puncture area of a patient; and
a guiding platform pivotably coupled to the base body and configured to assume different angles of inclination with respect to the base body, when the guiding platform is arranged at an angle of inclination of 0° with respect to the base body and the first contact surface of the base body rests on the puncture area, the guiding platform is pivotal between an angle of inclination of 0° and 180° with respect to the base body, when the guiding platform is arranged at an angle of inclination of 0° with respect to the base body and the second contact surface of the base body rests on the puncture area, the guiding platform is pivotal between an angle of inclination of 0° and 90° or 0° and −90° with respect to the base body.

2. The medical device according to claim 1, further comprising a support plate coupled to the guiding platform and configured to support a part of the puncture device, the support plate slidable longitudinally on the guiding platform between a first longitudinal position and a second longitudinal position, the support plate, guiding platform and base part being configured such that when either of the first and second contact surfaces of the first base part rests on the puncture area and the part of the puncture device is supported on the support plate, the pointed element is not in the puncture position when the support plate is in the first longitudinal position and is in the puncture position when the support plate is in the second longitudinal position.

3. The medical device according to claim 1, wherein the guiding platform is configured to be fixed at the different angles of inclination.

4. The medical device according to claim 1, wherein the base body includes a third contact surface that is configured to be supported on a surface of a probe of an ultrasound device.

5. The medical device according to claim 1, wherein an end portion of the guiding platform is coupled to a rotating shaft that extends through at least a portion of the base body.

6. The medical device according to claim 5, wherein the base body includes a plurality of spaced-apart notches into which the rotating shaft is configured to be movable between and secured to alter the puncture position.

7. The medical device according to claim 5, wherein the base body includes first and second supports that are arranged spaced-apart and parallel to one another, the rotating shaft extending between the first and second supports.

8. The medical device according to claim 6, wherein the base body includes first and second supports that are arranged spaced-apart and parallel to one another, the rotating shaft extending between the first and second supports.

9. The medical device according to claim 5, wherein the base body comprises first and second supports that are attached to one another and arranged in a L-shape, the rotating shaft extending from one of the first and second supports.

10. The medical device according to claim 6, wherein the base body comprises first and second supports that are attached to one another and arranged in a L-shape, the rotating shaft extending from one of the first and second supports.

11. The medical device according to claim 1, wherein the guiding platform pivotal between a first position in which the guiding platform is arranged inside a housing of the base body, and a second position in which the guiding platform is pivoted partially outside of the housing of the base body.

12. The medical device according claim 1, further comprising a rod extending between the base body and the guiding platform, the rod being configured to fix the angle of inclination of the guiding platform with respect to the base body.

13. The medical device according claim 6, further comprising a rod extending between the base body and the guiding platform, the rod being configured to fix the angle of inclination of the guiding platform with respect to the base body.

14. The medical device according claim 12, wherein the rod is attachable to different points of the base body.

15. The medical device according to claim 2, wherein a part of the support plate resides inside a recess of the guiding platform.

16. The medical device according to claim 2, wherein the guiding platform includes a longitudinal channel defined by a base, a first sidewall and a second sidewall facing the first sidewall, at least a portion of the support plate being housed in the channel and longitudinally guided by surfaces of the first and second sidewalls.

17. The medical device according to claim 16, wherein the at least portion of the support plate is slidable on a surface of the base.

18. The medical device according to claim 2, wherein the guiding platform comprises an element for measuring a distance traveled by the support plate.

19. The medical device according to claim 18, wherein the measuring device is a ruler.

20. The medical device according to claim 2, wherein the support plate includes a cavity that is configured to house the part of the puncture device.
